(19) United States
(12) Patent Application Publication
(10) Pub. No.: US 2008/0314745 A1
(43) Pub. Date: Dec. 25, 2008

(54) Biosensor and its production

(75) Inventors: Ingo Neubert, Norderstedt (DE); Maren Klose, Seevetal (DE)

Correspondence Address: NORRIS, MCLAUGHLIN & MARCUS, 875 THIRD AVE, 18TH FLOOR, NEW YORK, NY, 10022, US

(73) Assignee: tesa AG, Hamburg (DE)

(21) Appl. No.: 12/020,008

(22) Filed: Jan. 25, 2008

(30) Foreign Application Priority Data
Apr. 17, 2007 (DE) 10 2007 018 383.8

(51) Int. Cl. G01N 33/487

(52) U.S. Cl. 204/403.01

(57) ABSTRACT

Biosensor for analyzing biological fluids having:
a functional base layer A1 provided with electrical interconnects and at least partially with an analytical detection reagent,
a functional layer A3 and
a double-sided pressure-sensitive adhesive tape A2 which joins the functional layers A1 and A3 and forms a measuring channel whose lid is formed by functional layer A3 and whose base is formed by functional layer A1,
   the functional layer A3 having at least one hydrophilic and at least one hydrophobic region, the hydrophobic and hydrophilic regions being disposed such that they at least partially form an inside of the wall of the measuring channel formed by functional layer A2, and one hydrophilic region of the functional layer A3 ends flush with a front edge of the measuring channel, from which the biological fluid is supplied.

The present invention relates to a biosensor, and to its production, by means of which biological fluids such as blood, urine, saliva or cell fluid, for example, are analyzed. In the text below, a biosensor, more generally called microfluidic devices, also embraces analytical test strips, which are also known as medical diagnostic strips.

In modern medical diagnostics an ever greater number of biosensors are being used. With the aid of these biosensors it is possible to analyze, for example, biological fluids such as blood, urine and saliva on the one hand for pathogens, incompatibilities, DNA activity or enzyme activity and on the other hand for levels of glucose, cholesterol, proteins, ketones, phenylalanine or enzymes.

On the biosensors, detection reactions or reaction cascades take place. For this purpose the biological test fluid must be transported to the reaction site or to the various reaction sites. The modern biosensors therefore consist of at least one microchannel or micro-channel system through which the test fluid is transported. The microchannels typically have a height and width of 5 to 1500 μm. Transport within the channels is accomplished by means of capillary forces or centrifugal forces. The results of the detection reactions are usually read optically or electrochemically.

One of the first patents in the technical field of test strips appeared back in 1964. U.S. Pat. No. 1,073,596 A describes a diagnostic test and the test strips for analyzing biological body fluids, especially for determining blood sugar. The diagnostic test functions via the determination of a color change which is triggered by an enzyme reaction.

Determining a change in concentration of a dye (calorimetric method) is still a method used today in the determination of blood sugar by means of diagnostic test strips. It involves reaction of the enzyme glucose oxidase/peroxidase with the blood sugar. The hydrogen peroxide formed then reacts with the indicator, such as O-tolidines, for example, which leads to a color reaction. This color change can be monitored by calorimetric methods. The degree of changing color is directly proportional to the blood sugar concentration. In that case the enzyme is located on a woven fabric.

This method is described for example in EP 0 451 981 A1 and WO 93/03673 A1.

The modern development of diagnostic test strips aims to shorten the measuring time between the application of blood to the test strip and the appearance of the result. The measuring time, or the time between the application of the blood to the diagnostic measuring strip and the display of the result, is dependent not only on the actual reaction time in the enzymic reaction and in the follow-on reactions, but likewise, to a considerable extent, on the time taken for the blood to be transported within the diagnostic strip from the blood application point to the reaction site, in other words to the enzyme.

One of the ways in which the measuring time is shortened is by the use of hydrophilicized woven or nonwoven fabrics, as in U.S. Pat. No. 6,555,061 B, to transport the blood more quickly to the measuring region (enzyme). The measuring method is identical with that described in EP 0 451 981 A1. In the construction of the diagnostic strip a double-sided standard adhesive tape, Scotch® 415, is used. Surface-modified woven fabrics having a wicking effect for the biological fluid are described in WO 93/03676 A1, WO 03/067252 A1 and US 2002/0102739 A1. In the latter application, plasma treatment of the woven fabric produces a blood transport rate of 1.0 mm/s. However, when woven fabrics are used for transporting the biological test fluid such as blood, for example, a chromatography effect is observed: that is, the individual constituents such as cells are separated by the liquid constituents. The chromatography effect is exploited explicitly in WO 03/008933 A2 for the separate analysis of the blood constituents.

An onward development from the colorimetric measuring method is the electrical determination of the change in oxidation potential at an electrode coated with the enzyme. This method and a corresponding diagnostic test strip are described in WO 01/67099 A1. The diagnostics test strip is constructed by the printing of different functional layers such as interconnects, enzyme and hotmelt adhesive to the base material of, for example, polyester. Subsequently, an otherwise unspecified hydrophilic film is laminated on by thermal activation of the adhesive. The purpose of the hydrophilic film, here again, is to accelerate the transport of the blood to the measuring cell.

With this construction there is no need for woven or nonwoven fabric to transport the blood. The advantage of this construction and the advantage of the new measuring method is that the blood sugar level can be measured with a very much smaller volume of blood, around 5 to 10 μl, and in a shorter measuring time.

U.S. Pat. No. 5,997,817 A describes an electrochemical biosensor in which the transport of the biological fluid is realized likewise by way of a hydrophilic coating. The coating in question is ARCARE 8586 (not available commercially) from Adhesive Research Inc. The transport of the biological fluid is evaluated in a specific capillary test of which, however, no further details are given.

DE 102 34 564 A1 describes a biosensor which is composed of a planar sensor or test strip and a compartmentalized reaction-and-measurement chamber attachment which is produced by embossing of a PVC film. The measurement chamber attachment is composed of a very specific embossed design comprising sample application duct, measurement chamber, sample arrest duct and sample collection space. The embossed depth of this compartmentalization amounts to 10 to 300 μm. The sample application duct and the measurement chamber are furnished with a woven hydrophilic fabric or with a surfactant coating for the transport of the biological fluid. This specific capillary geometry accelerates, retards or arrests the transport of the test fluid in the biosensor.

A very similar electrochemical sensor is described in U.S. Pat. No. 5,759,364 A1. The sensor is composed of a printed base plate and an embossed cover film of PET or polycarbonate. The measuring space here is coated with a polyurethane ionomer for accelerated fluid transport.

DE 102 11 204 A1 describes a flow-through measuring cell for the continuous determination of glucose. The measuring cell is composed of a planarly structured film which forms a small inlet duct and a substantially larger outlet duct, the two ducts opening into one another via a defined angle.

Further typical applications include, for example, biosensors (for example U.S. Pat. No. 5,759,364 A1) and blood sugar test strips (for example WO 2005/033698 A1, U.S. Pat. No. 5,997,817 A1). In this connection see also B. Grunding, Landbauforschung Völkerode (2002) SH 241:63-70.

With all of these systems it is important to make it possible for the transport of fluid to be checked or influenced. On the one hand, for the reason of short measuring times, a very rapid transport of fluid is desired. On the other hand, it is necessary to retard or arrest the transport of fluid. This is important for an exact detection reaction.

Various studies on the topics of capillarity and liquid transport in capillaries are to be found in the literature. The capillary pressure, or the ascension of a column of liquid in a capillary, is dependent on the surface tension of the liquid, the viscosity of the liquid, the wetting angle and the diameter of the capillary, and is determined in accordance with the following formula (equation 1 (eq. 1)):

$$h = \frac{4 * {γ}_{l} * cos θ}{g ( {ζ}_{l} - {ζ}_{g} ) * d} \qquad eq . 1$$

h—ascension or depression
γₗ—surface tension of the liquid
ζₗ—density of the liquid
ζ_{g}—density of the gas (air)
g—acceleration due to gravity
θ—contact angle (wetting angle)
d—internal diameter of the capillary

FIG. 1 illustrates equation 1.

From this equation it is evident that the capillary forces increase as the capillary diameter d goes down. A reduction in flow rate in a capillary can therefore be achieved by increasing the cross section of a microchannel. A further important parameter affecting the flow rate of a given liquid is the surface tension of the inside of the channel, whereas for a given biological fluid it is not possible to vary the viscosity as a parameter.

If the wetting angle between liquid and capillary wall is very small, capillary ascension occurs, meaning that the liquid rises in the capillary. For a contact angle of >90°, however, the result is capillary depression, and the level of the liquid in the capillary is below the external liquid level (W. Bohl “Technische Strömungslehre”, 13th, revised and expanded edition, Vogel Verlag, June 2005, ISBN: 3834330299, page 37f.).

In the literature there are numerous studies on surface tension and on the phenomenon of the wettability of solids.

The wetting of a solid by a liquid is described by Young's equation (eq. 2) (in this connection see FIG. 2)

γₗ*cos θ=γₛ−γₛₗ eq. 2

θ—contact angle (wetting angle)
γₗ—surface tension of the liquid
γₛ—surface tension of the solid
γₛₗ—interfacial tension between the liquid and the solid

If the surface tensions of the solid and of the liquid are significantly different, a contact angle θ>>90° is obtained. The surface of the solid is not wetted by the liquid. In the range from 90° to 20°, wetting of the solid's surface occurs. At contact angles θ<20°, the surface tensions between liquid and solid are very similar, and the surface of the solid is wetted very well by the liquid. At contact angles θ<<20°(θ˜0°), the liquid spreads out on the surface of the solid (see “Die Tenside”, Kosswig/Stache, Carl Hanser Verlag, 1993, ISBN 3-446-16201-1, page 23).

The literature describes the use of surfactants, which the skilled person knows as substances with interface activity, for improving wettability. Surfactants are molecules or polymers which consist of an apolar/hydrophobic portion (tail) and a polar/hydrophilic group (head). To improve the wettability of surfaces, the surfactants are added to the aqueous liquid. The surfactant brings about a reduction in the surface tension of the aqueous liquid at the interfaces (liquid-solid and liquid-gaseous). This effect of improving the wettability of the surfaces is measurable in a reduction in the contact angle and in the reduction in the surface tension of the liquid. The skilled person distinguishes between anionic, cationic, amphoteric and nonionic surfactants. The hydrophobic tail of surfactants may consist of linear or branched alkyl, alkylbenzyl, perfluorinated alkyl or siloxane groups. Possible hydrophilic head groups are anionic salts of carboxylic acids, phosphoric acids, phosphonic acids, sulphates, sulphonic acids, cationic ammonium salts or nonionic polyglycosides, polyamines, polyglycol esters, polyglycol ethers, polyglycol amines, polyfunctional alcohols or alcohol ethoxylates (see also Ullmann's Encylcopedia of Industrial Chemistry, Vol. A25, 1994, page 747).

The surface may likewise be modified by plasma treatment. For that purpose the surface is treated with a plasma in a vacuum. Through the introduction of gases or organic substances it is possible to achieve selective adjustment of the surface properties. In this way both hydrophilic and hydrophobic layers can be produced on the surface. The application of this method is described in WO 03/086960 A1.

WO 01/02093 A2 describes the use of a film having a microstructured surface for the checking of fluid transport.

A number of publications mention the use of hydrophilic materials such as woven fabrics (DE 30 21 166 A1), membranes (DE 198 49 008 A1) and films (EP 1 358 896 A1, WO 01/67099 A1), but without any more detailed characterization of the hydrophilic coatings.

DE 198 15 684 A1 describes an analytical aid consisting of a zone with capillary activity, an adhesive tape diecut and a top film with capillary activity. The top film of capillary activity possesses hydrophilic surface properties which are achieved by vapor coating of the top film with aluminium and by subsequent oxidation.

There are various patents which concern themselves with the subject of a hydrophilic coating. Examples include US 2005/0084681 A1, EP 1 647 568 A1, US 2002/0110486 A1 and EP 1 394 535 A1.

Likewise known in the literature are a number of examples in which selectively hydrophilic and hydrophobic regions are utilized for the purpose of controlling the transport of fluid. In U.S. Pat. No. 6,601,613 B2 the fluid transport is controlled by the capillary's geometry. Another possibility mentioned, though not described in any more detail, is the use of surfactants or hydrophobic polymers.

U.S. Pat. No. 6,969,166 B2 describes a modified surface having two different contact angles. The modification is accomplished by digital printing (inkjet printing) of hydrophobic polymers on the basis of fluoropolymers or silicone polymers and of hydrophilic polymers. In digital printing a halftone print is produced, that is, the printed areas are not coherent but instead consist of individual printed dots.

It is an object of the present invention to provide a biosensor which in accordance with the requirements is suitable for the analysis of biological fluids and which specifically is able on the one hand rapidly to transport the biological fluid into the measuring channel and on the other hand to carry out defined arrest of the transport of the biological fluid into the measuring channel. It must also be ensured here that the properties, and especially the wetting properties and transport properties in the measuring channel of the biosensor, are retained even after a long storage time.

This object is achieved by means of a biosensor as described hereinbelow. The invention further embraces the possibility for use of the biosensor of the invention inter alia in medical sensors or diagnostic strips for analyzing biological fluids.

The invention accordingly provides a biosensor by means of which biological fluids are analyzed, comprising at least the following layers:
a functional layer A1 which constitutes the base layer of the biosensor and is provided with electrical interconnects and at least partially with an analytical detection reagent,
a functional layer A3 and
a double-sided pressure-sensitive adhesive tape A2 which joins the functional layers A1 and A3 to one another and in which a measuring channel is provided whose lid is formed by the functional layer A3 and whose base is formed by the functional layer A1,
   the functional layer A3 having at least one hydrophilic and at least one hydrophobic region, the hydrophobic and hydrophilic regions of the functional layer A3 being disposed such that they at least partially form an inside of the wall of the measuring channel formed by the functional layer A2, and one hydrophilic region of the functional layer A3 finishes directly with the front edge of the measuring channel, the entry orifice 2, from which the biological fluid is supplied to the medical biosensor.

### BRIEF DESCRIPTION OF THE DRAWINGS

The invention will now be described in greater detail with reference to the drawings, wherein:

FIG. 1 is an illustration depicting the ascension of a column of liquid in a capillary tube in contact with a liquid;

FIG. 2 is an illustration depicting the wetting of a solid by a liquid as described by Young's equation;

FIG. 3 is a schematic depicting the construction of one biosensor embodiment according to the present invention;

FIG. 4 is another schematic depicting the construction of one biosensor embodiment according to the present invention;

FIG. 5 is another schematic depicting the construction of one biosensor embodiment according to the present invention;

FIG. 6 is another schematic depicting the construction of one biosensor embodiment according to the present invention; and

FIG. 7 is another schematic depicting the construction of one biosensor embodiment according to the present invention.

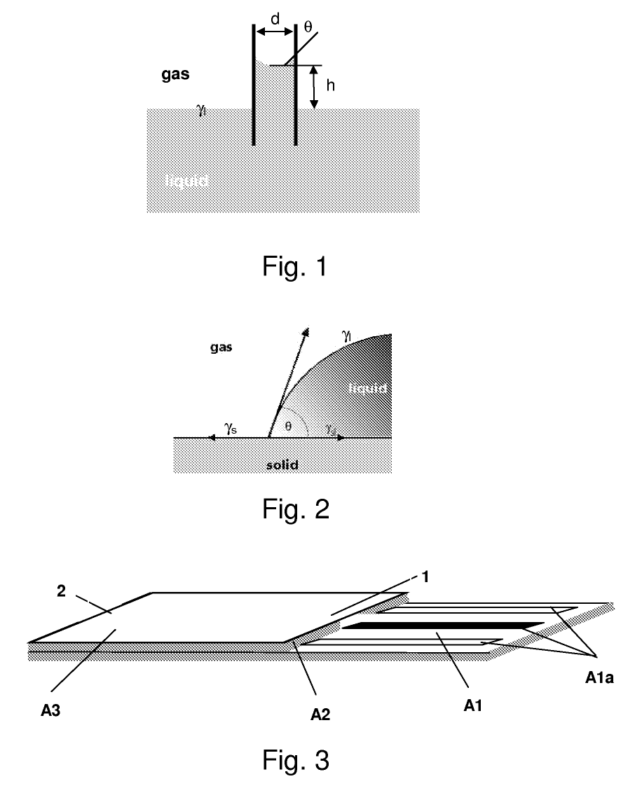

In one advantageous embodiment of the invention on the functional layer A3 there are two or more hydrophilic and two or more hydrophobic regions which are disposed adjacently, more particularly in alternation adjacently, and which contact one another preferably at the borderlines.

In one variant of the invention the hydrophobic and advantageously also the hydrophilic region on the functional layer A3 form a partial but uniform coherent area.

With further preference on the functional layer A3 the hydrophobic region is applied partially over the hydrophilic region, which advantageously is coated over the whole area of the functional layer A3.

The layer thickness of the hydrophobic region is advantageously 0.1 μm to 30 μm. The layer thickness of the hydrophilic region is preferably not more than 3 μm and advantageously not more than 1.5 μm.

With further preference both the hydrophilic regions and the hydrophobic regions are applied in the form of lines, more particularly in a printing process.

The line width of the hydrophilic regions is 0.3 to 10 mm and/or that of the hydrophobic regions is 0.5 to 10 mm, advantageously 1 to 3 mm.

The hydrophobic regions of the functional layer A3 are preferably composed of a coating which has a surface tension of not more than 30 mN/m, a contact angle with water of greater than 90°, preferably greater than 95°, and a release value with the test adhesive tape tesa® 7475 of less than 50 cN/cm. With particular preference the coating is composed of a release varnish based on a fluoropolymer or on a silicone polymer, which forms an impervious coating. The hydrophobic coating may be crosslinked preferably by means of UV radiation.

When the biosensor of the invention is used, the filling of the measuring channel with the biological test fluid is very quick. Surprisingly for the skilled person, the transport of the biological fluid can be arrested completely after the measuring channel has been filled. This is necessary for the electrochemical concentration measurement of a biological analyte, the determination of blood sugar content being an example. Particularly surprising and unforeseeable is the fact that the fluid front can be arrested completely by the hydrophobic region despite the fact that the hydrophobic region is located only at one of the four walls of the channel. Countering the arrest are the capillary forces and also the surface-active substances (surfactants) which are introduced into the fluid by the hydrophilic region and which dissolve in the test fluid. These results are unforeseeable owing to the great complexity of the parameters influencing the capillary forces such as, for example, capillary geometry, the different surface tensions of the various walls, different polarity and the viscosity of the biological test fluid, and the influence of surface-active substances.

FIG. 3 shows by way of example the construction of a biosensor having a measuring channel 1, which is formed by a diecut of a double-sided pressure-sensitive adhesive tape and constitutes the functional layer A2. In this example the measuring channel A2a is formed by two parallel walls of the diecut, the measuring channel being open at both ends and in 2 forming the front edge or entry orifice of the measuring channel, from which the sample fluid is introduced into the measuring channel. The functional layer A2 with the measuring channel 1 is laminated from one side with the functional layers A1 and A3. Hence the functional layer A1 likewise forms a wall of the measuring channel. Located on the functional layer A1 are electrical interconnects Ala and also a partial coating of the detection reagent or the enzyme (not shown on the drawing). In terms of dimensions the functional layer A1 is longer than the functional layers A2 and A3, and thus projects at least on one side. This allows access to the electrical interconnects A1a that are applied here, and it is possible to establish electrical contact with the read device. The measuring channel 1 is bounded from the opposite side by the functional layer A3 as a further wall. The functional layer A3 is equipped with at least one hydro-philic and one hydrophobic region, which point to the inside of the measuring channel and thus at least partially form a wall. In this arrangement the hydrophilic region A3a borders the entry orifice 2 of the measuring channel 1.

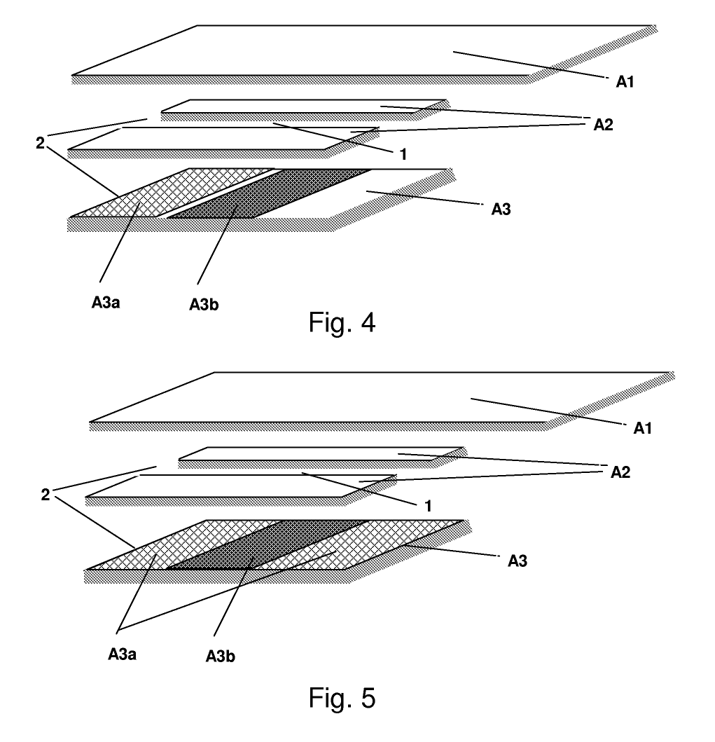

In FIG. 4, which is constructed like FIG. 3, the functional layer A3 is seen to have in each case one hydrophilic region A3a and one hydrophobic region A3b, which are disposed adjacently. In FIG. 5 the hydrophobic region A3b is applied partially to the full-area hydrophilic region A3a. In both figures the hydrophilic region A3a and the hydrophobic region A3b are positioned over the measuring channel in such a way that they form at least partially a part of the inside wall of the measuring channel and in such a way that the hydrophilic region A3a finishes with the entry orifice 2 of the measuring channel 1.

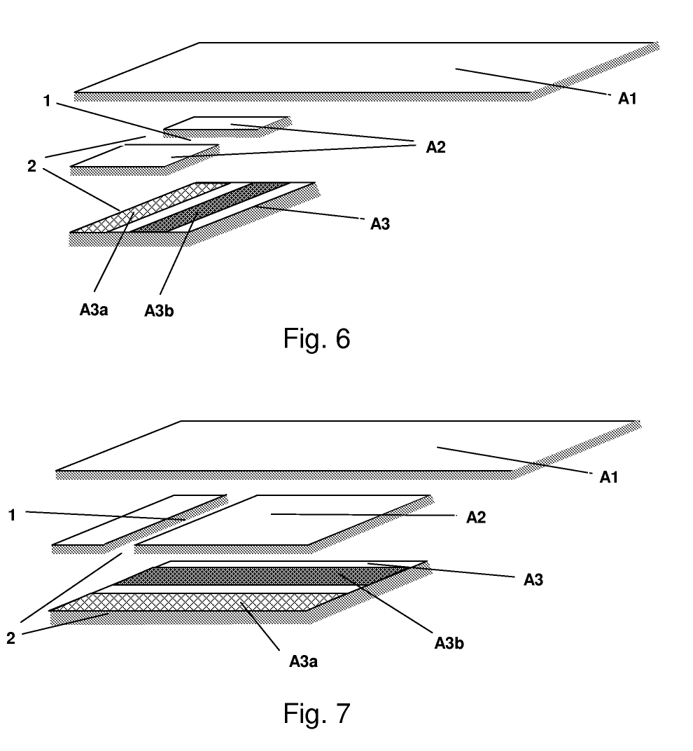

FIG. 6 shows an advantageous embodiment in which the measuring channel and also, accordingly, the hydrophilic and the hydrophobic region, however, have very small dimensions. As a result of this it is possible to realize a test strip with very low test fluid volumes.

FIG. 7 shows a preferred embodiment in which the measuring channel is disposed transversely to the test strip. The hydrophilic region A3a and the hydrophobic region A3b here again form part of the inner wall of the measuring channel 1, the hydrophilic region A3a finishing with the entry orifice 2 of the measuring channel 1.

This recitation should not be considered conclusive; instead, within the bounds of the invention, further designs are included.

The biosensor of the invention for investigating analytes in biological fluids operates preferably in accordance with an electrochemical (amperometric) measuring method. Preference is given in this context to the detection of glucose in human blood, or blood sugar determination. The biosensor of the invention is composed of the functional layer A1. This constitutes the base layer of the biosensor and is functionalized as follows. The base film, consisting for example of PVC, paper, polycarbonate or, preferably, polyester, with a thickness range of 200 to 500 μm, is provided with electrical interconnects. An electrochemical biosensor typically requires working electrode, counterelectrode and, if appropriate, reference electrode. The electrical interconnects can be applied in a printing process such as screen printing, for example, to the base film. This is done using conductive pastes, which for example may be conductive carbon, graphite or silver pastes. Depending on the construction there may be insulating layers, likewise applied by printing, between the various interconnect layers. Alternatively the base film can also be laminated, vapor-coated or sputter-coated with a conductive layer of copper, silver, gold or aluminium, for example. In this case the interconnects are obtained in a downstream operation by etching or by a laser ablation process (in this regard see WO 006/074927 A1). Applied to the working electrode and counterelectrode is the enzyme or enzyme mixture needed for the detection reaction and comprising, for example, glucose oxidase/peroxidase and a redox mediator, for example ferrocene or derivatives. The citable prior art includes WO 2005/033698 A1, WO 2005/101994 A1, U.S. Pat. No. 6,541,216 B1 and EP 1 253 204 A1.

The pressure-sensitive adhesive tape of the functional layer A2 may be made not only of one or more layers of a pressure-sensitive adhesive transfer tape (pressure-sensitive adhesive tape without carrier film), which may be laminated with carrier films, but also of a double-sided pressure-sensitive adhesive tape consisting of a carrier film coated on both sides with the pressure-sensitive adhesive.

The adhesives and the application rates of adhesive on the top and bottom sides of the pressure-sensitive adhesive tape may be identical, but can also be selected differently, in order to meet the particular requirements.

The sum of the application rate of the layers of adhesive in the functional layer A2 is in one advantageous embodiment not more than 70 g/m² and preferably not more than 50 g/m².

With further preference the pressure-sensitive adhesive tape is composed of a polyester carrier film coated on each side advantageously with not more than 35 g/m² and more preferably with not more than 25 g/m² of a pressure-sensitive adhesive.

The characteristic quality of the biosensor of the invention is a function of the combination between the pressure-sensitive adhesive tape and the functional layers A1 and A3, the adhesive or pressure-sensitive adhesive of the pressure-sensitive adhesive tape exhibiting a high level of cohesion or shear strength, respectively. The use of a pressure-sensitive adhesive with a high shear strength is necessary in order to avoid residues of adhesive in the biosensor production process and so to make the production process efficient. On the other hand, the pressure-sensitive adhesive must have sufficient bond strength to prevent delamination of the functional layers and also to prevent the test fluid running down between the pressure-sensitive adhesive tape and the functional layer.

The high shear strength of the pressure-sensitive adhesive is expressed in a high holding power of more than 10 000 min at 70° C. under a weight load of 1000 g and also in a shear deformation after 15 min at 40° C. under a load of 500 g of less than 130 μm and preferably less than 80 μm.

The pressure-sensitive adhesive tape forms the measuring channel, which is advantageously produced in a diecutting process. With particular advantage the pressure-sensitive adhesive tape forms a measuring channel comprising two parallel walls, the measuring channel being open towards both ends. Two further walls of the measuring channel are formed by the functional layer A1 and A3.

Suitability for preparing the adhesive of the pressure-sensitive adhesive tape with the properties described is possessed by copolymers or copolymer blends of acrylate monomers, or styrene block copolymers with, for example, ethylene, propylene, butylene, butadiene, hexene and/or hexadiene as comonomers.

In the preferred version the pressure-sensitive adhesive of the pressure-sensitive adhesive tape is composed of one or more copolymers of at least the following monomers:
c1) 70% to 100% by weight of acrylic esters and/or methacrylic esters or their free acids, with the following formula:

CH₂═CH(R₁)(COOR₂),
where R₁ is H and/or CH₃ and R₂ is H and/or alkyl chains having 1 to 30 C atoms.

Here as well it is possible for the parent monomer mixture to have had
c2) up to 30% by weight of olefinically unsaturated monomers with functional groups added to it as a further component.

In one very preferred design use is made for the monomers c1) of acrylic monomers which comprise acrylic and methacrylic esters with alkyl groups consisting of 4 to 14 C atoms, preferably 4 to 9 C atoms. Specific examples, without wishing to be restricted by this enumeration, are n-butyl acrylate, n-pentyl acrylate, n-hexyl acrylate, n-heptyl acrylate, n-octyl acrylate, n-nonyl acrylate, lauryl acrylate, stearyl acrylate, behenyl acrylate, and their branched isomers such as, for example, t-butyl acrylate and 2-ethylhexyl acrylate.

Further classes of compound which may likewise be added in small amounts under c1) are methyl methacrylates, cyclohexyl methacrylates, isobornyl acrylate and isobornyl methacrylates.

In one very preferred embodiment use is made for the monomers c2) of vinyl esters, vinyl ethers, vinyl halides, vinylidene halides, vinyl compounds with aromatic rings and hetero-cycles in α position.

Here again mention may be made of a number of examples, without the enumeration being considered conclusive:
vinyl acetate, vinyl formamide, vinylpyridine, ethyl vinyl ether, vinyl chloride, vinylidene chloride and acrylonitrile.

In a further very preferred embodiment use is made for the monomers c2) of monomers having the following functional groups:
hydroxyl, carboxyl, epoxy, acid amide, isocyanato or amino groups.

In one advantageous variant there are for c2) acrylic monomers corresponding to the general formula

CH₂═CH(R₁)(COOR₃),
where R₁ is H or CH₃ and the radical R₃ represents or constitutes a functional group which supports subsequent UV crosslinking of the pressure-sensitive adhesive—which for example, in one particularly preferred embodiment, possesses an H donor effect.

Particularly preferred examples for component c2) are hydroxyethyl acrylate, hydroxypropyl acrylate, hydroxyethyl methacrylate, hydroxypropyl methacrylate, allyl alcohol, maleic anhydride, itaconic anhydride, itaconic acid, acrylamide and glyceridyl methacrylate, benzyl acrylate, benzyl methacrylate, phenyl acrylate, phenyl methacrylate, t-butylphenyl acrylate, t-butylphenyl methacrylate, phenoxyethyl acrylate, phenoxyethyl methacrylate, 2-butoxyethyl methacrylate, 2-butoxyethyl acrylate, dimethylaminoethyl methacrylate, dimethylaminoethyl acrylate, diethylaminoethyl methacrylate, diethylaminoethyl acrylate, cyanoethyl methacrylate, cyanoethyl acrylate, glyceryl methacrylate, 6-hydroxyhexyl methacrylate, N-tert-butylacrylamide, N-methylol-methacrylamide, N-(buthoxymethyl)methacrylamide, N-methylolacrylamide, N-(ethoxymethyl)acrylamide, N-isopropylacrylamide, vinylacetic acid, tetrahydrofurfuryl acrylate, β-acryloyloxypropionic acid, trichloroacrylic acid, fumaric acid, crotonic acid, aconitic acid, dimethylacrylic acid, where this enumeration should not be understood as being conclusive.

In a further preferred embodiment use is made for component c2) of aromatic vinyl compounds, where the aromatic nuclei are preferably composed of C₄ to C₁₈ and may also include heteroatoms. Particularly preferred examples are styrene, 4-vinylpyridine, N-vinylphthalimide, methylstyrene, 3,4-dimethoxystyrene, 4-vinylbenzoic acid, where this enumeration should not be considered as being conclusive.

For preparing the polyacrylate pressure-sensitive adhesives it is advantageous to carry out conventional free-radical polymerizations or controlled free-radical polymerizations. For the polymerizations which proceed by a radical mechanism it is preferred to use initiator systems which in addition contain further free-radical initiators for the polymerization, more particularly thermally decomposing free-radical-forming azo or peroxo initiators. In principle, however, all typical initiators familiar to the skilled person for acrylates are suitable. The production of C-centered free radicals is described in Houben Weyl, Methoden der Organischen Chemie, Vol. E 19a, pages 60 to 147. These methods are preferentially employed in analogy.

For the advantageous ongoing development no additives at all such as tackifying resins or plasticizers are added to the polyacrylate pressure-sensitive adhesive of the pressure-sensitive adhesive tape. Although such additives increase the bond strength they may considerably reduce the shear strength of the pressure-sensitive adhesive and so may lead to residues of adhesive on the slitting tools during the operation of slitting the biosensors.

In summary the preferred embodiment of the pressure-sensitive adhesive tape features a polyacrylate pressure-sensitive adhesive which is manufactured by coextrusion or by coating from the melt, solvent or dispersion. Particular preference is given to comma bar coating of the polyacrylate pressure-sensitive adhesive from a suitable solvent or solvent mixture.

The pressure-sensitive adhesive tape of the invention may optionally comprise a carrier film coated on both sides with the pressure-sensitive adhesive (PSA). Carrier materials used are the typical carrier materials familiar to the skilled person, such as films of polyester, polyethylene, polypropylene, oriented polypropylene, polyvinyl chloride, more preferably films of polyethylene terephthalate (PET). This enumeration should not be considered as being conclusive; instead, in the context of the invention, other films are included. In order to enhance the adhesion of the adhesive to the carrier film and hence to avoid residues of adhesive on the slitting tool during the operation of slitting the biosensors it is possible to use a primer layer between carrier film and pressure-sensitive adhesive or, preferably, to perform a physical surface treatment such as flaming, corona or plasma on the carrier film.

Advantageously, diecuts having a diecut shape suitable for the application are produced from the pressure-sensitive adhesive tape. The diecut from the pressure-sensitive adhesive tape in this case forms the measuring channel and, accordingly, the functional layer A2. The diecut shape preferably forms a cutout comprising two parallel walls, which is open at both ends. Other diecut designs are possible. The pressure-sensitive adhesive tape diecuts are produced using the customary methods such as flat-bed diecutting, rotary diecutting, ultrasonic slitting, water-jet slitting or laser slitting. Production of the diecuts requires a very high level of precision, in the μm range. The diecut produced from the pressure-sensitive adhesive tape can be laminated, immediately after the diecutting operation, to the functional layer A3, thereby allowing the combination of the diecut of the pressure-sensitive adhesive tape (functional layer A2) and the functional layer A3 to be supplied directly to the biosensor production process. It is also possible, however, for the pressure-sensitive adhesive tape and the functional layer A3 to be supplied separately to the biosensor production process and to be laminated to one another only once they are there. The diecuts are preferably produced in the form of continuous rolls, in a so-called roll-to-roll operation, without being separated. In this case only the future measuring channel is cut out. With particular preference, in the same operation of the diecutting process, as described, the functional layer A3 is laminated onto the functional layer A2. In a corresponding inline operation, in other words in the same operating step, the narrow printed hydrophobic and hydrophilic lines of the functional layer A3 can be positioned optimally onto the diecuts of the functional layer A2. Precise positioning is vital for the functioning of the biosensor of the invention. It is particularly advantageous if the hydro-phobic and hydrophilic lines of the functional layer A3 are likewise printed inline in the diecutting operation, and the lamination of the functional layers A2 and A3 is likewise carried out inline in this operation. An alternative possibility is for the entire production process of the biosensors, of the production of the interconnects and printing with the enzyme layer (functional layer A1), the production of the diecuts of the functional layer A2 and the printing of the hydrophobic and hydrophilic lines of the functional layer A3, and also the lamination of these 3 functional layers, to take place in one and the same operation. The separation or singularization of the biosensors takes place typically in a separate slitting operation.

The functional layer A3 has at least one hydrophilic and one hydrophobic region. The hydrophilic region ensures on the one hand that the test fluid flows into the measuring channel. For this purpose the hydrophilic region must extend up to the front edge of the entry orifice of the measuring channel. If this is not the case it is impossible for the test fluid to be transported into the measuring channel, particularly if it is a fluid having a relatively high viscosity such as blood, for example. On the other hand, the hydrophilic region is likewise important for the very rapid transport of the test fluid into the measuring channel, so that measurement can be commenced as quickly as possible in order to be able to realize an extremely short measuring time. The measuring times in blood sugar test strips on the market at present are 3 sec.

The hydrophilic region is applied either over the full area or partially to the functional layer A3. Full-area application takes place advantageously in a coating process. Examples of suitable coating processes include spray coating, halftone roller application, Mayer bar coating, multi-roll application coating, condensation coating or else printing processes. Partial coating takes place preferably with a printing process such as screen printing or flexographic printing. In this case the viscosity of the coating solution is adapted to suit the printing process. This is typically done using a polymer as binder. A digital printing process such as inkjet printing is less suitable for the present inventions, since with this printing process the printed area produced is not coherent but instead takes the form of separate printed dots. This may adversely affect the properties of the hydrophilic region.

The hydrophilic regions of the functional layer A3 are preferably composed of a coating or of an imprint having a surface tension of at least 60 mN/m and having a contact angle with water of less than 30°.

According to one further advantageous embodiment of the invention the hydrophilic region of the functional layer A3 is produced from a coating varnish which is composed of polyvinyl alcohol as binder, a surfactant and water and which preferably prior to drying has a viscosity of 50 to 500 mPa*s and more preferably of 80 to 200 mPa*s.

It is advantageous anyway if the hydrophilic region of the functional layer A3 comprises at least one surfactant, which is preferably an anionic surfactant and more preferably a surfactant based on a sulphosuccinic ester salt. Preferably the surfactant is used in a fraction of 0.5% to 10% by weight, more preferably 1% to 5% by weight based on the binder.

The hydrophilic coating is preferably composed, accordingly, of at least one surfactant and may likewise include further additions such as, for example, a polymer as binder. The surfactant is critically responsible for the hydrophilic properties. Surfactants which can be used include compounds comprising linear or branched alkyl, alkylbenzyl, perfluorinated alkyl or siloxane groups with hydrophilic head groups, such as anionic salts of carboxylic acids, phosphoric acids, phosphonic acids, sulphates, sulphonic acids, sulphosuccinic acid, cationic ammonium salts or nonionic polyglycosides, polyamines, polyglycol esters, polyglycol ethers, polyglycol amines, polyfunctional alcohols or alcohol ethoxylates. This selection is an exemplary enumeration and does not represent any restriction of the inventive concept to the surfactants specified.

By way of example the following suitable surfactants may be specified:
nonionic fatty alcohol ethoxylated surfactants, for example Tego Surten® W111 from Evonik AG or Triton® X-100 and Tergitol® 15-S from Dow Chemicals Inc.
nonionic fluoro surfactants, for example Fluorad® FC-4430 and FC-4432 from 3M Inc., Zonyl® FSO-100 from DuPont Inc. and Licowet® F 40 from Clariant AG
nonionic silicone surfactants, for example Q2-5211 and Sylgard® 309 from Dow Corning Inc., Lambent® 703 from Lambent Technologie Inc. and Tegopren® 5840 from Evonik AG
ionic alkyl sulphate salt, for example Rewopol® NLS 28 from Evonik GmbH
ionic sulphosuccinic salts, for example Lutensit® A-BO from BASF AG or Rewopol® SB DO from Evonik GmbH

Particular preference is given to using an ionic sulphosuccinic salt and with very particular preference sodium diisooctylsulphosuccinate (CAS number 577-11-7) as surfactant. The ionic sulphosuccinic salts are particularly suitable on account of their very good wetting behavior with very good ageing resistance and low mobility. The very good wetting behavior is exhibited in a surface tension of at least 60 mN/m and in a contact angle with water of less than 30°. Nor is there any change in the wetting behavior after a long storage time, which can be simulated by accelerated ageing at elevated temperatures, for example 70° C. Low mobility is necessary in the surfactant in order to avoid transfer of the surfactant to guide rolls in the production and processing operation.

The coating for the hydrophilic region preferably likewise comprises at least one polymer. The polymer here acts as a binder for this surfactant-containing coating. As the polymer it is possible to use all of the film-forming binders that are known from the printing inks industry. Advantageously the binder used will be a polymer having polar functional groups such as, for example, hydroxyl, carboxyl, ether, ester, amine, amide groups. Suitable binders that may be mentioned, by way of example and without restriction, are homopolymers or copolymers such as polyvinylpyrrolidone, polyvinylbuteral polyesters, polyacrylate, polyacrylic acid, polyvinyl acetate, polyvinyl alcohol, polyacrylamide, polyamide, polyethylene glycol, polypropylene glycol, cellulose derivatives. Advantageously water is used as a solvent for the hydrophilic coating, and advantageously, therefore, the binder is water-soluble.

In one preferred variant of the coating varnish of the invention a polyvinyl alcohol is used as binder. Polyvinyl alcohols are prepared from polyvinyl acetate by hydrolysis of the acetate functionality. The properties of the polyvinyl alcohols can be controlled on the one hand via the molecular weight of the polymer and on the other hand via the degree of hydrolysis. Preference is given to using a polyvinyl alcohol having a degree of hydrolysis of >85 mol % and more preferably of >95 mol %. As an example of this class of polymer, mention may be made of Mowiol® from Kuraray or Polyviol® from Wacker Chemie GmbH. Surprisingly it is possible, through a combination of 0.5% to 10% by weight and preferably 1% to 5% by weight of a sulphosuccinic salt as surfactant, based on the binder, preferably polyvinyl alcohol, and of a binder, to produce a hydrophilic coating varnish having reproducible properties. The hydrophilic properties of this coating varnish are unchanged even after a long storage time of 10 weeks at 70° C. With particular preference the aqueous polyvinyl alcohol solution has a dynamic viscosity of 50 to 500 mPa*s, more preferably of 80 to 200 mPa*s, which produces an optimum between thickness of application, printability and anchorage. The optimum adaptation of viscosity of the print varnish is very important in order to achieve a good print outcome. A good print outcome is manifested in the present invention by a uniformly narrow printed line with a width of 0.3 to 10 mm and advantageously of 0.5 to 3 mm and also by a uniform coating thickness of not more than 3 μm and advantageously of not more than 1.5 μm. A uniform coating thickness is important more particularly for adhesive bonding with the functional layer A2. If the printed hydrophilic coating line is too thick, it may be the case that the test fluid, owing to the difference in height, will run beneath the functional layer A2 at the interface. This greatly threatens the functional capacity of the biosensor.

The hydrophilic coating may likewise comprise further additives such as organic dyes or inorganic pigments, ageing inhibitors and/or fillers (in this regard see “Plastics Additives Handbook”, sections “Antioxidants”, “Colorants”, “Fillers”, Carl Hanser Verlag, 5th edition).

The hydrophilic region of the functional layer A3 may be applied in the form of a full-area or partial coating, with a solvent, to the carrier material. Solvents used are water, alcohols, ethanol, or higher-boiling alcohols such as n-butanol or ethoxyethanol, ketones such as butanone, esters such as ethyl acetate, alkanes such as hexane, toluene or mixtures of the aforementioned solvents.

The hydrophobic region of the functional layer A3 ensures that the transport of the test fluid in the measuring channel is arrested at a defined location, namely the hydrophobic region. This is important for an exact detection reaction. The hydrophobic coating is preferably composed of a release varnish, also called release lacquer. Typical release varnish coatings are produced on the basis of fluoro polymers or silicone polymers. These release varnish coatings are notable for their hydrophobic character and for their low surface tension. Particularly suitable as a hydrophobic coating of the invention are fluorinated polymers or polysiloxane-based polymers. Polysiloxane release varnish coatings are produced, for example, by the companies Wacker, Rhodia or Dow Corning. Solvent-based coatings, emulsion-based coatings or 100% systems are suitable. These polysiloxane coatings are typically crosslinked by means of a free-radical reaction, addition reaction or condensation reaction. Crosslinking takes place either thermally during the drying of the coating, or, with particular preference, by UV radiation of a 100% system. Mention may be made, for example, of UV-crosslinking, printable silicone release varnishes Syl-Off UV® from Dow Corning, Silicolease, UV Poly 205 from Bluestar Silicones, UV 9200 SGS from GE Bayer Silicones, Tego RC 1403 from Evonik GmbH and UVX00192, UAAS0032 or UAS00107 from XSys GmbH.

For the functional capacity of the hydrophobic region, namely the arresting of the transport of the test fluid in the measuring channel, it is important for the hydrophobic region to form a coherent area on the functional layer A3. If this is not the case, the test fluid cannot be reliably arrested, and there are breakthroughs of fluid. Therefore, a coating method which produces, for example, halftone dots, digital printing for example, is not suitable for the application. The hydrophobic region of the functional layer A3 is applied advantageously in a printing process and with particular preference in flexographic printing. In the context of coating, a uniform coating thickness is important especially for adhesive bonding to the functional layer A2. If the printed hydrophobic coating line is too thick, the difference in height at the interface may result in the test fluid running beneath the functional layer A2. Underruns are formed. This greatly jeopardizes the functional capacity of the biosensor. The width chosen for the hydrophobic lines is as narrow as possible, since the anchorage of the functional layer A2 on this hydrophobic varnish is not ensured and hence at this point there is a weakening of the laminate. On the other hand the line width must be sufficiently wide to ensure the functional capacity, in other words the arresting of the test fluid. In one advantageous embodiment of the present invention the hydrophobic region of the functional layer A3 is formed by uniformly narrow printed lines having a width of 0.3 to 10 mm and advantageously of 0.5 to 3 mm and also by a uniform coating thickness of 0.1 to 3 μm and advantageously of 0.5 to 1.5 μm. Likewise suitable for the hydrophobic coating is a release varnish based on fluorinated polymers. As well as polysiloxanes, mention may also be made, for example, of hydrophobic coatings comprising polymers or copolymers of vinylidene fluoride hexafluoropropene, hexafluoroisobutylene and tetrafluoroethene.

The hydrophobic coating may likewise comprise organic dyes or inorganic pigments, ageing inhibitors and/or fillers (in this regard see “Plastics Additives Handbook”, sections “Antioxidants”, “Colorants”, “Fillers”, Carl Hanser Verlag, 5th edition).

The base material used for the functional layer A3 comprises typical carrier materials that are familiar to the skilled person, such as films of polyethylene, polypropylene, oriented polypropylene, polyvinyl chloride, polyesters and, with particular preference, polyethylene terephthalate (PET). The films in question may be single films, coextruded films or laminated films, in unoriented or monoaxially or biaxially oriented form. This enumeration is exemplary and not conclusive. The surface of the films may be microstructured as a result of suitable methods such as embossing, etching or laser treatment, for example. The use of laminates, woven or nonwoven fabrics or membranes is likewise possible. For better anchorage of the coating, the carrier materials may be chemically or physically pretreated by the standard methods—mention may be made, for example, of corona treatment or flame treatment. To promote adhesion it is likewise possible to prime the carrier material with, for example, PVC, PVDC, polyurethanes or thermoplastic polyester copolymers. The thickness of the carrier film is 12 to 350 μm and preferably 50 to 150 μm.

### Test Methods

### Surface Tension and Contact Angle Measurement

The measurement of the contact angle with water and of the surface tension on solid surfaces takes place in accordance with EN 828:1997 using a G2/G402 instrument from Kruss GmbH. The surface tension is determined by the Owens-Wendt-Rabel&Kaeble method, by measuring the contact angle with deionized water and diiodomethane. The values are obtained in each case from the averaging of four results. For the purpose of the measurement, full-area drawdowns of the respective coating solution on a 100 μm PET film are produced in the laboratory.

### Functional Test

To assess the transport characteristics of an aqueous test fluid, a capillary test is carried out. This is done by placing the application orifice of the biosensor into a test fluid consisting of deionized water and 1% by weight of naphthol red. The transport of the test fluid in the hydrophilic region, and the arrest of the transport on reaching the hydrophobic region, are observed by means of a video camera. The testing channel is left in the test fluid for several minutes in order to ensure that the fluid is not, over time, transported on beyond the border of the hydrophobic region.

The channel test is also carried out after the biosensors under test have been stored at 23° C., 40° C. and 70° C., in order to test the ageing resistance and storage stability.

As the test fluid, use is likewise made of biological fluids such as blood. Biological fluids such as blood, however, are less suited as test fluid, since they are subject to fluctuations in properties. For example, the viscosity of blood fluctuates very sharply, being dependent on the haematocrit value.

### Dynamic Viscosity Measurement

The viscosity of the coating varnish is measured using a Rheometrix DSR 200N at a shear rate of 10 1/s with a cone/plate system having a diameter of 50 mm.

### Bond Strength

The peel strength (bond strength) was tested in a method based on PSTC-1. A strip of the pressure-sensitive adhesive tape 2 cm wide is adhered to the test substrate, such as a ground steel plate or a PET plate, for example, by applying the tape and running a 5 kg roller back and forth over it five times. The plate is clamped in and the self-adhesive strip is pulled by its free end in a tensile testing machine under a peel angle of 180° at a speed of 300 mm/min; the force required in order to pull the strip is measured. The results are reported in N/cm and are averaged over three measurements. All of the measurements were conducted at room temperature.

### Shear Deformation

A strip of the pressure-sensitive adhesive tape 1 cm wide is adhered to a polished steel plaque (test substrate) over a length of 5 cm, by applying the strip to the plaque and rolling a 2 kg roller back and forth over it three times. Double-sided adhesive tapes are lined on the reverse with a 50 μm aluminium foil. The test strip is reinforced with a PET film 190 μm thick and then cut off flush using a fixing device. The edge of the reinforced test strip projects 1 mm beyond the edge of the steel plaque. The plaques are equilibrated for 15 minutes under test conditions (40° C., 50% relative humidity) in the measuring instrument, but without a load. Then the 500 g test weight is hung on, producing a shearing stress parallel to the bond surface. A micro-travel recorder records the shear travel in graph form as a function of time. The shear deformation reported is the shear path after 15 minutes of weight loading.

### Release Forces

This test determines the release behavior of release varnish coatings relative to the standardized test adhesive tape tesa® 7475.

Characterization of the test adhesive tape tesa® 7475

| | | | Thickness | Bond |
|---|---|---|---|---|
| Carrier | Adhesive | Liner | (without liner) | strength/steel |
| PVC film, | 95 g/m² | release | 0.135 mm | 12.5 N/cm |
| 40 μm, white | acrylate | paper | | |

For the purpose of the measurement, full-area drawdowns of the respective coating solution on a 100 μm PET film are produced in the laboratory. For the determination of the release forces, 20 mm wide strips of the test adhesive tape tesa® 7475 are adhered to the release varnish coating under test. This is followed by storage under a 20 kg block weight at 70° C. for 24 h. After conditioning for 2 h at 23° C. and 50% relative humidity, a tensile testing machine with a take-off speed of 0.3 m/min is used to determine the force required to peel the test adhesive tape at an angle of 180° C. from the release varnish coating under test. This measurement is carried out along the general lines of PSTC-4B.

### Thickness Measurement

The thickness measurement or the hydrophilic and hydrophobic coating applied, is made by way of the optical method of reflectometry with a NanoCalc 2000 UV/Vis from Mikropack and a microscope from Leitz. This method utilizes the difference in the refractive indices of the different materials. The refractive index of the particular coating must be determined prior to measurement. For the PET base film a refractive index of 1.46 is used.

The intention of the text below is to illustrate the invention by means of a number of examples, without wishing thereby to restrict the invention unnecessarily.

### EXAMPLES

### Example 1

A biosensor is produced by lamination from the functional layers A1, A2 and A3. The construction of the functional layer A1 is as follows: a 250 μm PET film, Hostaphan WO from Mitsubishi Polyesterfilm GmbH, is printed with the interconnects, using a conductive graphite paste E3455 from Ercon Inc. In the region of the measuring space, the reactive layer is then applied to the working electrode, said reactive layer consisting of the active component glucose dehydrogenase, the coenzyme NAD+, the mediator 1,10-phenanthroline, and a hydroxyethylcellulose binder. For the functional tests (fluid transport), the coating of the interconnects and of the reactive layer is omitted for the sake of simplicity.

For the functional layer A3, the 100 μm Hostaphan® RN 100 PET film from Mitsubishi Polyesterfilm GmbH is corona-pretreated on one side and then coated over its full area, using a halftone roller, with a solution consisting of 0.5% by weight of Rewopol® SB DO 75 (sodium salt of diisooctylsulphosuccinic acid), from Evonik GmbH, in ethanol. The coating is dried in a drying tunnel at 120° C. After drying, the thickness of application is 25 nm. In a second operation, this hydrophobic coating is printed by the flexographic printing process with a hydrophobic line 1.2 mm wide. The hydrophobic coating used is the UVF00080 flexographic varnish (UV-crosslinking, free-radically crosslinking flexographic varnish, viscosity 300 mPa*s) from XSys GmbH with 5% by weight of UAS00107 (UV-drying, free-radically crosslinking silicone additive). The coating is cured by means of UV radiation. The coating has a thickness of 0.5 μm.

For the functional layer A2, first of all the pressure-sensitive adhesive is prepared. For this purpose, a reactor conventional for free-radical polymerization was charged with 8 kg of acrylic acid, 45 kg of n-butyl acrylate, 3 kg of t-butyl acrylate and 60 kg of acetone. After nitrogen gas had been passed through the reactor for 45 minutes with stirring the reactor was heated to 58° C. and 20 g of azoisobutyronitrile (AIBN, Vazo 6®, DuPont) were added. Subsequently the external heating bath was heated to 75° C. and the reaction was carried out constantly at this external temperature. After a reaction time of 1 h a further 20 g of AIBN were added. After 3 h and 6 h the mixture was diluted with 10 kg of acetone/isopropanol (97:3) each time. In order to reduce the residual initiators, after 8 h and after 10 h, 100 g portions of bis(4-tert-butylcyclohexanyl) peroxydicarbonate (Perkadox 16®, Akzo Nobel) are added each time. After a reaction time of 22 h the reaction is terminated and cooled to room temperature.

After the polymerization the polymer is diluted with isopropanol to a solids content of 25% and then blended with 0.3% by weight of polyisocyanate (Desmodur N 75, Bayer) with stirring. Subsequently the polymer solution is coated using a comma bar onto both sides of the 50 μm Hostaphan WO polyester carrier from Mitsubishi Polyesterfilms GmbH, which is pretreated by means of corona, with 12 g/m² in each case. The coating is dried in a drying tunnel at 120° C. A rotary punch is used to produce diecuts from the resulting pressure-sensitive adhesive tape, the diecuts having a cut-out measuring channel with dimensions of 1.2 mm×5 mm in accordance with FIG. 5. In the diecutting operation, with a precise fit, the functional layer A3 is laminated onto the diecuts of the functional layer A2 in such a way that the hydrophobic region is located at the point intended for it. The positioning is monitored via a camera system.

The lamination of the functional layer A1 to the assembly comprising functional layer A2 and A3, for the production of test specimens, is carried out by hand on the small scale. For production operation, this lamination would likewise take place inline in the diecutting operation. The individual biosensors are cut from the roll material in a downstream operation.

As a result of the hydrophilic region of the functional layer A3, a high transport rate is achieved with an aqueous test fluid when the measuring channel is filled. The transport of fluid in the measuring channel comes to a standstill at the front edge of the hydrophobic region. Even after a further supply of test fluid there is no further transport beyond the edge of the hydrophobic line. The transport behavior of the test fluid in the biosensor specimens does not change even after storage at 40° C. or 70° C. for 6 weeks.

### Example 2

A biosensor is produced from the functional layers A1, A2 and A3 by lamination. Functional layer A1 is identical with that described in Example 1.

To produce the functional layer A3 the corona-pretreated PET film Hostaphan® RN from Mitsubishi Polyesterfilm GmbH, with a thickness of 190 μm, is printed by the flexographic printing process on a Nilpeter printing machine first with a hydrophobic line 2.0 mm wide and subsequently with a hydrophobic line 1.0 mm wide. The two lines run parallel to one another, and the distance between these two lines is 0.5 mm. The hydrophilic print varnish is dried using IR lamps and the hydrophobic print varnish is cured using UV radiation. The printed design corresponds to FIG. 4. The application thickness of the hydrophilic line is 0.5 μm, and the application thickness of the hydrophobic line is 0.6 μm. for the hydrophilic print varnish, 10 kg of Mowiol 4-98, Kuraray Specialities Inc., and 0.3 kg of Rewopol SB DO 75 from Evonik GmbH are dissolved in 60 kg of water with continual stirring. The resulting print varnish has a viscosity of 130 mPa*s. For the hydrophobic coating the UV release varnish UVX00192 (UV-drying, cationic UV release varnish, viscosity 270 mPa*s) from XSys GmbH is used.

The pressure-sensitive adhesive of the functional layer A2 is prepared as follows. A reactor conventional for a free-radical polymerization is charged with 28 kg of acrylic acid, 292 kg of 2-ethylhexyl acrylate, 40 kg of methyl acrylate and 300 kg of acetone/isopropanol (97:3). After nitrogen gas had been passed through the reactor for 45 minutes with stirring the reactor was heated to 58° C. and 0.2 kg of azoisobutyronitrile (AIBN, Vazo 6®, DuPont) were added. Subsequently the external heating bath was heated to 75° C. and the reaction was carried out constantly at this external temperature. After a reaction time of 1 h a further 0.2 kg of AIBN were added. After 3 h and 6 h the mixture was diluted with 150 kg of acetone/isopropanol (97:3) each time. In order to reduce the residual initiators, after 8 h and after 10 h, 0.4 kg portions of bis(4-tert-butylcyclohexanyl) peroxydicarbonate (Perkadox 16®, Akzo Nobel) are added each time. After a reaction time of 22 h the reaction is terminated and cooled to room temperature. After the polymerization the polymer is diluted with isopropanol to a solids content of 25% and then blended with 0.4% by weight of aluminium(III) acetylacetonate with stirring. Subsequently the polymer solution is coated using a comma bar onto both sides of the 50 μm Hostaphan WO polyester film from Mitsubishi Polyesterfilm GmbH, which is pretreated by means of corona. Drying takes place in a drying tunnel at 120° C. The coatweight per coating side is 12 g/m².

Diecuts are produced in accordance with Example 1 from the pressure-sensitive adhesive tape. The diecut is laminated inline with a functional layer A3, thereby allowing the printed hydrophilic and hydrophobic regions to be positioned exactly, the hydrophilic printed line finishing with the application orifice of the measuring channel, as depicted in FIG. 4.

The lamination of the functional layer A1 to the assembly comprising functional layer A2 and A3, for the production of test specimens, is carried out by hand on the small scale. As a result of the hydrophilic region of the functional layer A3, a high transport rate is observed with an aqueous test fluid when the measuring channel is filled. The transport of fluid in the measuring channel comes to a standstill at the front edge of the hydrophobic region. Even after a further supply of test fluid there is no further transport beyond the edge of the hydrophobic line. In the same way as for the biosensors produced in Example 1, the functional capacity is fully retained even after 6 weeks at 40° C. or 70° C.: in other words, even after storage, the test fluid flows very rapidly into the measuring channel and with equal reliability is arrested by the hydrophobic region.

### Example 3

A biosensor is produced from the functional layers A1, A2 and A3 by lamination. Functional layer A1 is identical with that described in Example 1.

To produce the functional layer A3, the PET film Hostaphan® RN 100 from Mitsubishi Polyesterfilm GmbH, with a thickness of 100 μm, is first coated with the primer NeoRez R650 (PU Primer), Neoresins Inc., and then printed by the flexographic printing process first with a hydrophobic line 1.0 mm wide and then with a hydrophobic line 1.0 mm wide. The two lines run parallel to one another, and the distance between these two lines is 0.4 mm. The hydrophilic print varnish is dried with IR lamps, and the hydrophobic print varnish is cured with UV radiation. The printed design corresponds to FIG. 6. The application thickness of the hydrophilic line is 0.5 μm, and the application weight of the hydrophobic line is 0.5 μm. The hydrophilic print varnish is prepared as described in Example 2. For the hydrophobic coating the UV release varnish Silcolease UV Poly 205 (UV-drying, cationic UV release varnish, viscosity 220 mPa*s) from Bluestar Sylicons S.A.S. is used, blended with 5% by weight of Flexonic Reflexblau from XSys GmbH.

The pressure-sensitive adhesive of the functional layer A2 is prepared as described in Example 2. The polymer solution is applied using a comma bar to both sides of a 75 μm thick polyester film, Hostaphan WO from Mitsubishi Polyesterfilm GmbH, which is pretreated by means of corona beforehand. Drying takes place in a drying tunnel at 120° C. The coatweight per coating side is 25 g/m².

From the pressure-sensitive adhesive tape, in accordance with Example 1, diecuts are produced, but in this example the diecut measuring channel has the dimensions 0.8 mm×2 mm. The diecut is laminated inline with a functional layer A3, so that the printed hydrophilic and hydrophobic regions are positioned exactly, the hydrophilic printed line finishing with the application orifice of the measuring channel, as shown in FIG. 6. Positionally accurate lamination is made easier as a result of the coloured hydrophobic line.

The lamination of functional layer A1 with the assembly of functional layer A2 and A3 for the production of test specimens is carried out by hand on the small scale.

In the same way as the abovementioned exemplary biosensors, this biosensor as well exhibits very good filling times of the measuring channel with the test fluid and reliable arresting of the test fluid, after storage of the biosensor at 70° C.

### COMPARATIVE EXAMPLES

### Comparative Example 1

The functional layers A1 and A2 are produced in the same way as in Example 2.

For the production of the functional layer A3, the corona-pretreated PET film Hostaphan® RN 100 from Mitsubishi Polyesterfilm GmbH, with a thickness of 100 μm, is printed by the flexographic printing process with a hydrophilic line 1.0 mm wide. The hydrophilic coating used is an aqueous/ethanolic solution (in a 4:1 ratio), consisting of 0.5% by weight of Tegopren 5840 from Goldschmidt GmbH, 25% by weight of Luvitec K30 from BASF AG and 0.3% by weight of Irganox 1010 from Ciba AG. The viscosity of the coating solution is 290 mPa*s. The coating is dried thermally in the printing operation. The application thickness of the hydrophilic printed line is 3.1 μm.

As described in Example 2, a biosensor is produced from the functional layers A1, A2 and A3 in such a way that the hydrophilic printed line finishes with the application orifice of the measuring channel.

The biosensor initially exhibits rapid transport and filling of the measuring channel with the test fluid. However, the transport of the test fluid does not stop after it reaches the end of the hydrophilic printing. The test fluid flows completely through the measuring channel until the channel is completely filled. Furthermore, owing to the high application thickness of the hydrophilic line, running of the test fluid underneath between the functional layer A2 and A3, at the edge of the line, is observed. After storage of the biosensor at 70° C., the fluid transport function is lost after just a few weeks, and the measuring channel can no longer be filled. This ageing behavior cannot be adequately counteracted even by adding ageing inhibitors.

### Comparative Example 2

The functional layers A1 and A2 are produced in the same way as in Example 2.

For the production of the functional layer A3, the corona-pretreated PET film Hostaphan® RN 100 from Mitsubishi Polyesterfilm GmbH, with a thickness of 100 μm, is printed by the flexographic printing process first with a hydrophobic line 2.0 mm wide and then with a hydrophobic line 1.0 mm wide. The two lines run parallel to one another, and the distance between these two lines is 0.2 mm. For the hydrophilic printing, 10 kg of Mowiol 4-98 from Kuraray Specialities Inc. are dissolved in 60 kg of water with continual stirring. The resulting print varnish has a viscosity of 130 mPa*s. The coating is dried thermally in the printing operation. The hydrophobic coating used is the UV release varnish UVX00192 from XSys GmbH. The coating is cured by means of UV radiation. The application thickness of the hydrophilic line is 0.5 μm, and the application thickness of the hydrophobic line is 0.9 μm.

As described in Example 2, a biosensor is produced from the functional layers A1, A2 and A3 in such a way that the hydrophilic printed line finishes with the application orifice of the measuring channel, as depicted in FIG. 4.

The wetting properties of the hydrophilic coating are moderate, as is manifested in a relatively high wetting angle. The transport of the aqueous test fluid into the test channel is unreliable. The beginning of transport into the channel is often delayed or does not take place at all. Where there is transport of fluid into the channel, a high fluctuation in the transport rate is observed visually. However, this transport does then stop reliably at the hydrophobic line.

### Comparative Example 3

The functional layers A1 and A2 are produced in the same way as in Example 2.

For the production of the functional layer A3, the corona-pretreated PET film Hostaphan® RN 100 from Mitsubishi Polyesterfilm GmbH, with a thickness of 100 μm, is printed by the flexographic printing process first with a hydrophobic line 2.0 mm wide and then with a hydrophobic line 1.0 mm wide. The two lines run parallel to one another, and the distance between these two lines is 0.2 mm. For the hydrophilic print a print varnish as described in Example 2 is used. To produce the hydrophobic line, the UV printing ink UFZ 50129 from Siegwerk GmbH is used. The coating is cured by means of UV radiation. The application thickness of the hydrophilic line is 0.5 μm, and the application thickness of the hydrophobic line is 1.0 μm.

As described in Example 2, a biosensor is produced from the functional layers A1, A2 and A3 in such a way that the hydrophilic printed line finishes with the application orifice of the measuring channel, as depicted in FIG. 4.

The hydrophilic coating shows very good transport properties for the test fluid in the channel test (in analogy to Example 2). Arrest of liquid transport at the margin of the hydrophobic line, however, is not achieved; all that occurs is a brief slowing of transport of the fluid. After that the fluid front breaks through, and the fluid is transported to the end of the test channel.

Overview of the properties of the examples and counterexamples

| | | | | | | |
|---|---|---|---|---|---|---|
| | | | | Counter- | Counter- | Counter- |
| Unit | Example 1 | Example 2 | Example 3 | example 1 | example 2 | example 3 |

Functional layer A1

| | | | | | | | |
|---|---|---|---|---|---|---|---|
| Material | | PET | PET | PET | PET | PET | PET |
| Thickness | μm | 250 | 250 | 250 | 250 | 250 | 250 |

Functional layer A2

| | | | | | | | |
|---|---|---|---|---|---|---|---|
| Thickness of | μm | 75 | 75 | 125 | 75 | 75 | 75 |
| adhesive tape | | | | | | | |
| Thickness of | μm | 50 | 50 | 75 | 50 | 50 | 50 |
| carrier film | | | | | | | |
| Application of | g/m² | 2 × 12 | 2 × 12 | 2 × 25 | 2 × 12 | 2 × 12 | 2 × 12 |
| adhesive | | | | | | | |
| Bond strength to | N/cm | 2.5 | 1.8 | 3.2 | 1.8 | 1.8 | 1.8 |
| steel | | | | | | | |
| Shear | μm | 38 | 49 | 63 | 49 | 49 | 49 |
| deformation | | | | | | | |

Functional layer A3

| | | | | | | | |
|---|---|---|---|---|---|---|---|
| Material | | PET | PET | PET | PET | PET | PET |
| Thickness | μm | 100 | 190 | 100 | 100 | 100 | 100 |

Hydrophilic region

| | | | | | | | |
|---|---|---|---|---|---|---|---|
| Type of printing | | surfactant | PVAI + | PVAI + | PVP + | PVAI | PVAI + |
| | | | surfactant | surfactant | surfactant | | surfactant |
| Width of printed | mm | full area | 2.0 | 1.0 | 1.0 | 2.0 | 2.0 |
| lines | | | | | | | |
| Applied | μm | 0.025 | 0.5 | 0.5 | 3.1 | 0.5 | 0.5 |
| thickness | | | | | | | |
| Contact angle | ° | 21 | 19 | 19 | 19 | 58 | 19 |
| Surface tension | mN/m | 67 | 69 | 69 | 69 | 57 | 69 |

Hydrophobic region

| | | | | | | | |
|---|---|---|---|---|---|---|---|
| Type of printing | | varnish with | silicone | silicone | none | silicone | printing |
| | | silicone add. | varnish | varnish | | varnish | ink |
| | | | | with ink | | | |
| Width of printed | mm | 1.2 | 1.0 | 1.0 | — | 1.0 | 1.0 |
| lines | | | | | | | |
| Applied | μm | 0.5 | 0.6 | 0.5 | — | 0.9 | 1.0 |
| thickness | | | | | | | |
| Contact angle | ° | 102 | 109 | 108 | 73 (PET) | 109 | 82 |
| Surface tension | mN/m | 27 | 24 | 25 | 45 (PET) | 24 | 37 |
| Release value | cN/cm | 32 | 26 | 26 | — | 26 | 190 |

Biosensor

| | | | | | | |
|---|---|---|---|---|---|---|
| Design | FIG. 5 | FIG. 4 | FIG. 6 | FIG. 4 | FIG. 4 | FIG. 4 |

| | | | | |
|---|---|---|---|---|
| Function test | rapid fluid transport, fluid stops at | no arrest of | poor fluid | no arrest |
| | hydrophobic line | fluid | transport | of fluid |
| Function test | rapid fluid transport, fluid stops at | no fluid | — | — |
| after 6 weeks at | hydrophobic line transport | | | |
| 70° C. | | | | |

What is claimed is:

1. Biosensor for analyzing biological fluids, said biosensor comprising at least the following layers:
a functional layer A1 which constitutes a base layer of the biosensor and is provided with electrical interconnects and at least partially with an analytical detection reagent,
a functional layer A3 and
a double-sided pressure-sensitive adhesive tape A2 which joins the functional layers A1 and A3 to one another and in which a measuring channel is provided having a lid formed by the functional layer A3 and a base formed by the functional layer A1,
the functional layer A3 having at least one hydrophilic and at least one hydrophobic region, the hydrophobic and hydrophilic regions of the functional layer A3 being disposed such that they at least partially form an inside of the wall of the measuring channel formed by the functional layer A2, and
one hydrophilic region of the functional layer A3 ends at a front edge of the measuring channel, from which the biological fluid is supplied to the medical biosensor.

2. Biosensor according to claim 1,
wherein
on the functional layer A3 there are two or more hydrophilic and two or more hydrophobic regions which are disposed adjacent one another and which contact one another.

3. Biosensor according to claim 1,
wherein
the hydrophobic and optionally also the hydrophilic region on the functional layer A3 form a partial but uniform coherent area.

4. Biosensor according to claim 1,
wherein
on the functional layer A3 the hydrophobic region extends partially over the hydrophilic region, which optionally is coated over the whole area of the functional layer A3.

5. Biosensor according to claim 1,
wherein
the hydrophilic region on the functional layer A3 has a layer thickness that is not more than 3 μm and/or the hydrophobic region on the functional layer A3 has a layer thickness that is 0.1 μm to 3 μm.

6. Biosensor according to claim 1,
wherein
the hydrophilic and the hydrophobic regions on the functional layer A3 comprise print.

7. Biosensor according to claim 1,
wherein
the hydrophilic region on the functional layer A3 is in the form of lines having a line width of 0.3 to 10 mm and/or the hydrophobic region on the functional layer A3 is in the form of lines having a line width of 0.5 to 10 mm.

8. Biosensor according to claim 1,
wherein
the hydrophilic region or regions of the functional layer A3 have the following properties:
a surface tension of at least 60 mN/m and
a contact angle with water of less than 30°.

9. Biosensor according to claim 1,
wherein
the hydrophilic region of the functional layer A3 comprises a coat of varnish which is composed of polyvinyl alcohol as binder, a surfactant and water.

10. Biosensor according to claim 1,
wherein
the hydrophilic region of the functional layer A3 comprises at least one anionic surfactant.

11. Biosensor according to claim 1,
wherein
the hydrophobic region or regions of the functional layer A3 have the following properties:
a surface tension of not more than 30 mN/m,
a contact angle with water of greater than 95° and
a release value with an acrylate test adhesive tape of less than 50 cN/cm.

12. Biosensor according to claim 1,
wherein
the hydrophobic regions of the functional layer A3 is composed of a release varnish based on a fluoropolymer or a silicone polymer, each of which is optionally UV-crosslinkable.

13. Biosensor according to claim 1,
wherein
the measuring channel formed by the functional layers A1 and A3 and also by two parallel walls formed by the pressure-sensitive adhesive tape A2 is open at both ends.

14. Biosensor according to claim 1,
wherein
the pressure-sensitive adhesive tape of the functional layer A2 is composed of a pressure-sensitive adhesive whose shear deformation after 15 min at 40° C. under a load of 500 g is less than 130 μm.

15. A method comprising using a biosensor according to claim 1 in medical sensors or diagnostic strips for analyzing biological fluids.

16. A medical sensor comprising a biosensor according to claim 1.

17. A diagnostic strip comprising a biosensor according to claim 1.
